(19) United States
(12) Patent Application Publication
(10) Pub. No.: US 2010/0266646 A1
(43) Pub. Date: Oct. 21, 2010

(54) Assemblies, systems, and methods for skin treatment incorporating oxidized cellulose

(75) Inventors: Radim Dvorak, Tisnov (CZ); Julieann Doyle, Dublin (IE); Keith Real, Wicklow (IE)

Correspondence Address: RYAN KROMHOLZ & MANION, S.C., POST OFFICE BOX 26618, MILWAUKEE, WI, 53226, US

(73) Assignee: HemCon Medical Technologies, (IP) Ltd.

(21) Appl. No.: 12/657,922

(22) Filed: Jan. 29, 2010

(30) Foreign Application Priority Data
Jan. 30, 2009 (IE) 2009/0092

(51) Int. Cl. A61K 8/73; A61K 8/02; A61Q 19/08; C08B 1/00

(52) U.S. Cl. 424/401; 514/57; 428/357

(57) ABSTRACT

A refined oxidised cellulose (OC) material in various salt states having therapeutic treatments for skin. The micronized OC has a uniform particle size of the order of microns (0.001 to 0.050 mm). The OC is treated by further steps of oxidation, hydrolysis and refinement. The final product is chemically known as polyanhydroglucuronic acid, (PAGA). The salt versions of the OC are generally metal salts, such as sodium, calcium, zinc, copper, and silver.

### BACKGROUND OF THE INVENTION

The invention relates to a method for preparing a product comprising oxidized cellulose compounds, such as polyanhydroglucuronic acid (PAGA) and salts thereof, and to the uses thereof. The products are especially suitable for medicinal, pharmaceutical and cosmetic products. The present invention further relates to topical compounds and cosmetic products used to treat a person's skin, and, more specifically, relates to topical compounds that incorporate oxidized cellulose based products for the purpose of skin aging prevention through beneficial skin treatment properties.

Skin care and skin treatment is a growing field of technology. People are becoming more and more concerned with healthy skin and with combating the effects of aging, such as wrinkles and dry skin. Diminished collagen levels can lead to visible signs of facial aging (Lupo 2006). A wrinkle, very simply, is caused by the reduction of collagen (Lam, M. and Sullindro, M.). Aged skin is characterized by fine lines, wrinkles, dypigmentation and increased coarseness (Chiu, A. and Kimball, A. B.). Skin aging can be divided into two broad processes: chronological aging (intrinsic) and photo-aging (extrinsic). Chronological aging represents the structural, functional, and metabolic changes in the skin that parallel the aging and degenerative changes in other body organs. Dry and thin skin, fine wrinkles, abnormal blood vessels and age spots are symptoms of chronological aging.

Photo-aging is a separate process and largely involves damage to the collagen and elastin fibers in the skin. Overexposure to ultraviolet (UV) light causes oxidative damage which leads to wrinkles. UVA light is responsible for tanning, wrinkling, and melanoma, while UVB light is responsible for sunburn and basal and squamous cell carcinoma. Both contribute to aged looking skin. UV light may further damage skin by increasing the production of proteolytic enzymes that break down collagen. The reduction in collagen synthesis causes the appearance of wrinkles. From the age of 35 onwards there is a slowing of cellular turnover and regeneration. This leads to thinner skin, which in turn results in the dermal-epidermal interface becoming flatter. Consequently, there is less nutritional exchange between the dermis and the epidermis resulting again in slower metabolism of epidermal cells. Also by products of cellular metabolism including free radicals accumulate within skin cells as they can not be transported away efficiently (Lam, M. and Sullindro, M.).

The effects of oxidized cellulose (OC) in wound healing have been widely published. OC has been used as a specially treated form of surgical sponge which promotes clotting and is used as a temporary dressing. OC also has been used as a haemostat (i.e. a substance that controls bleeding) for over 50 years in the Western world. However, due to the fibrous nature of the material, it is limited in its consistency, effectiveness, and suitability for processing and is limited in its potential for other applications.

One form of OC, polyanhydroglucuronic acid, commonly referred to as PAGA, is effective in treatment of bleeding and hemorrhaging. PAGA accelerates wound healing, but its mode of action remains largely ambiguous. A possible reason for the wound healing effect of PAGA and its derivatives could be attributable to collagen. Collagen is a key extra-cellular matrix protein secreted by fibroblast cells, which offers a support structure to provide turgidity and support to the skin. Collagen forms the connective tissue, which is located beneath the dermis. However, when tissues are disrupted following injury, the normal function of collagen as a support protein is changed slightly. Collagen is needed to repair the defect; therefore fibroblasts deposit more collagen. Excess collagen comes to the site of injury and is involved in the wound healing process. This deposited collagen becomes cross-linked and organized during the final remodeling stage of wound healing and becomes the new extra-cellular matrix for the healed wound (Diegelmann, R. and Evans, M. 2004).

Besides proteins, polysaccharides represent the most widespread biopolymers found in the biosphere. As an example, up to 10 metric tons per year of cellulose, a 1,4 β D-glucane, is synthesized in nature. Other α and β glucanes bound e.g. by 1,2; 1,3; 1,4 and 1,6; or 1,2 and 1,4 glycosidic bonds in the main chain, mostly of microbial origin, gain increasing importance with ongoing research in the field. It is the presence of glucuronic acid units in the polymeric chain of the oligosaccharides or polysaccharides that, together with their molar mass and type of the principal glycosidic bond, constitutes the basis of their immunostimulative, antitumorous, anticoagulative, or else haemostyptic effects (1).

Glucuronoglucanes can preferably be prepared by relatively specific selective oxidation of the primary alcoholic group at the C₆ carbon atom of the glucopyranosic unit of natural polysaccharides by nitrogen oxides, the C₁ aldehydic group of the basic unit being protected by the glycosidic bond.

A variety of methods have been disclosed for preparing glucuronoglucanes and glucuronanes from natural glucanes, using the oxidative effects of NO_{χ}, either in the gaseous form (2, DE 0941282; DE 0967144), in a nonpolar reaction environment of inert liquids such as hydrogenated hydrocarbons (USSR SU 937462; U.S. Pat. No. 4,347,057; EP 0492990), or in a polar environment of aqueous solutions of acids such as HNO₃, H₃PO₄ or their mixtures with HSO₄, wherein NOₓ, is mostly generated directly in the oxidation liquor via dosed introduction of reducing substances such as, notably, NaNO₂ (GB 709684; CS AO 185366; GB 1593513; (3), (4)), or the reaction environment is created by introducing liquid NOₓ into aqueous HNO₃ (U.S. Pat. No. 4,100,341).

A disadvantage of these processes relates to the fact that their oxidative effects on the glucane molecule are non-uniform and only relatively specific, in that besides creation of carboxyl groups of the uronic type of C₆ carbon of the glucopyranosic unit, other types of successive reactions (such as formation of ONO₂ and NO groups on C₆) and secondary reactions (such as formation of COOH and other oxidized groups on end carbons C₁ and C₄, and notably on C₂ and C₃ carbons) do occur. In accordance with numerous publications (5, 6, 7, 8), extensive testing of polyanhydroglucuronic acids prepared by the action of NOₓ has led us to the conclusion that, besides carboxyl groups on C₆ carbon, several other aldehydes, ketones, and their condensation products are formed that have fundamental influence on the stability of the polyanhydroglucuronic acid product.

In recent years ((9, 10) and U.S. Pat. No. 6,127,573) a new method for oxidation of polysaccharides, called the TEMPO method, wherein the oxidation on the C₆ carbon by sodium hypochlorite is catalysed by a 2,2,6,6-tetramethylpiperidine-1-oxyl radical in the presence of bromide ions. A disadvantage of this method, similar to oxidation by NOₓ, is again the high product non-homogeneity due to heterogeneous reaction, notably in crystalline regions.

It is evident from the above that the preparation of stable PAGA product having required physical and chemical characteristics, destined for pharmaceutical and cosmetic use, is in no way a simple matter.

In health care practice one often encounters cases of capillary bleeding occurring during injuries or related to surgical interventions. The healing of the wounds frequently depends on attaining rapid homeostasis and creation of coagulum, to especially serve as a protection of the wound against infection. Application of D glucurono-1,4 β D-glucane, the so-called oxidized cellulose, as a non-toxic resorbable local haemostat to arrest bleeding from surface injuries or parenchymatous organs, osseous bleeding, and in general wherever use of conventional styptic means may be difficult or slow in functioning and less effective, has proved especially effective in similar cases.

Other disadvantages of the known methods described above are the non-uniform degree of both oxidation and degradation of individual polysaccharide particles or fibers, non-uniform content of bound nitrogen and other destabilizing sites in the macromolecule, as well as broad distribution of their molecular masses, altogether factors which can result in non-uniformity in resorbtion in the organism on applying the product as a haemostatic agent or in binding other substances or drugs such as anaesthetics, antibiotics or cytostatics.

WO98/33822 describes a method for preparing stable polyanhydroglucuronic acid with controlled physicochemical properties adapted to the intended use, thus reducing or fully suppressing deficiencies of conventional products manufactured as well as broadening the potential scope of applications thereof. A lot of the deficiencies described above are overcome by WO98/33822. Stabilized microdispersed PAGA is prepared with a reduced degree of crystallinity, its copolymers with anhydroglucose, and salts thereof, with a high degree of purity. The stable microdispersed PAGA prepared has easily controllable physicochemical characteristics.

Any improved method for the preparation of an oxidized cellulose product would have wide application.

### SUMMARY OF THE INVENTION

The present invention discloses a refined oxidised cellulose (OC) in various salt states. The micronized OC has a uniform particle size of the order of microns (0.001 to 0.500 mm). The OC is treated by further steps of oxidation, hydrolysis and refinement. The final product is chemically known as polyanhydroglucuronic acid, (PAGA). The salt versions of the OC are generally inorganic salts, such as ammonium, sodium, calcium, zinc, copper, and silver or organic salts, such as triethanol amine (TEA).

These salt compounds have been determined to have therapeutic effects for skin treatment. Most notable, the sodium salt of OC (Na—OC salt), has been found to have efficacy for skin hydration, as an antioxidant and most commercially important as a stimulator of collagen expression in the skin.

The present invention also provides methods of using and delivering the OC salts. These and other areas of importance and significance will become apparent from the following description.

### BRIEF DESCRIPTION OF THE DRAWINGS

FIG. 1 is a perspective view of a human's arm.

FIG. 2 is a cross-sectional view of the various layers of skin of the arm.

FIG. 3 is a further cross-sectional view of various layers of skin of the arm, showing a damaged outer layer of the skin, thereby allowing moisture to escape through the upper layer of the skin.

FIG. 4 is another cross-sectional view of various layers of the skin of the arm, with the outer layer of the skin depicted as being undamaged and retaining moisture within the skin.

FIG. 5 is a perspective view of a solution applied to a human's arm, with the solution comprising an oxidized cellulose material according to the present invention.

FIG. 6 provides a perspective view of a monitoring device being applied to the arm shown in FIG. 5 to determine the transepidermal water loss (TEWL) through the skin of the arm.

FIG. 7 graphically depicts the moisture retention of a compound containing 2% of the oxidized cellulose material developed according to the present invention, with the depicted values comparing the compound to a dry control compound.

FIG. 8 graphically depicts the moisture retention of a compound containing 5% of the oxidized cellulose material developed according to the present invention, with the depicted values comparing the compound to a dry control compound.

FIG. 9 graphically depicts the antioxidant status of compounds containing various amounts of the oxidized cellulose material developed according to the present invention, with the depicted values compared to other antioxidant materials currently used.

FIG. 10 graphically depicts the antioxidant status of further compounds containing various amounts of the oxidized cellulose material developed according to the present invention, with the depicted values compared to other antioxidant materials currently used.

FIG. 11 graphically depicts the amount of collagen secretion from fibroblasts incubated with oxidized cellulose material developed according to the present invention, compared to a control group.

FIG. 12 graphically depicts the amount of collagen secretion from fibroblasts incubated with a 0.4% w/v Na-PAGA material.

FIG. 13 provides a perspective view of a potential delivery system for the oxidized cellulose material of the present invention.

FIG. 14 provides a perspective view of another potential delivery system for the oxidized cellulose material of the present invention.

FIG. 15 provides a perspective view of yet another potential delivery system for the oxidized cellulose material of the present invention.

FIG. 16 provides a perspective view of a further potential use of the oxidized cellulose material of the present invention, incorporating the material into a nanostructured thin film.

FIG. 17 is a schematic representation of a process used to prepare the oxidized cellulose according to the present invention.

FIG. 18 is a graph showing the relative percentage of particle size distribution for a number of batches prepared using the process of the invention as determined by means of MASTERSIZER (Malvern Instruments Ltd. Ser. No. 34,044-02, dispersant ethanol).

FIG. 19 is a graph showing the difference between the particle size distribution of a product prepared using the process of the invention in comparison to a product prepared using two different versions of oxidative hydrolysis processes. (referred to as Comparative Method I and II).

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

I. Overview

The present invention contemplates products and methods for skin care and skin treatment. The products are generally salts of oxidized cellulose (OC), or other OC derivatives such as esters or amides. Most notably, the metal salts of PAGA of the present invention have shown therapeutic qualities, such as hydration of dry skin, as antioxidants for skin care, and collagen expression of the skin. Section II discusses the hydrating effects and the antioxidant effect of the compounds of the present invention, and section III discusses the effects of the present invention with respect to collagen expression. Discussion of hydration of the skin is pertinent to both the discussions in sections II and III, and will generally be discussed in both sections. Section IV presents various delivery methods for the present invention. Section V describes a process for producing OC derivatives of the present invention.

II. Hydrating and Antioxidant Effects of Polyanhydroglucuronic Acid (PAGA)

The term “antioxidant” literally means “against oxidation”. An antioxidant can neutralize a free radical by donating one of its electrons without jeopardizing its own chemical stability. In the human skin, antioxidants are normally present in concentrations to deal with physiologically produced oxidative stresses. Notably, these antioxidants decrease in concentration with age. The reason for antioxidants in the skin is that reactive oxygen species (ROS) can damage important skin components such as lipids and collagen. UV light also causes the formation of ROS. Examples of free radicals are the superoxide anion (O₂⁻) hydroxyl (OH⁻) and nitric oxide (NO⁻). The term ‘reactive oxygen species’(ROS) indicates not only free radicals based on oxygen, but also some non-radical by-products of oxygen, like hydrogen peroxide (H₂O₂) and singlet oxygen (¹O₂).

It has been shown that as a result of UVA/B irradiation of human skin equivalents, antioxidants are depleted in a dose-dependent manner and lipid and protein oxidation products are formed accordingly. If oxidative stress overwhelms the skin, e.g. from UV irradiation, the concentration of dermal antioxidants can decrease with an associated increase in the formation of ROS and associated dermal damage.

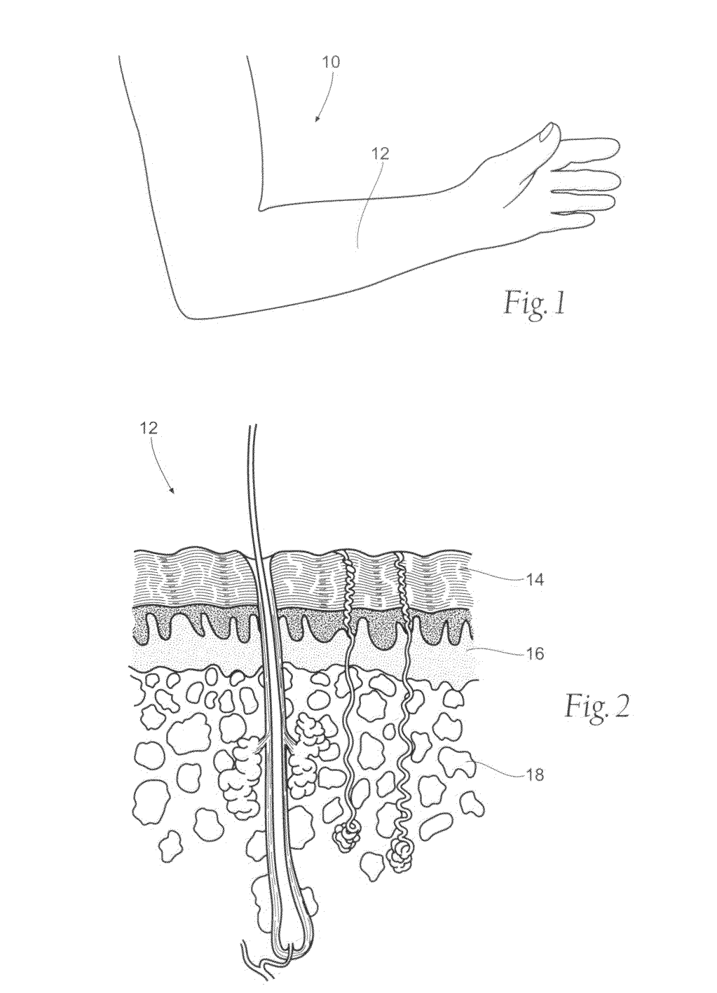

FIG. 1 depicts a human arm 10, being covered with skin 12. The arm 10 should be considered a typical arm of a typical person. The various compounds and applications that are applied, as discussed below, will be applied to a typical person's skin. The arm 10 and the skin 12 are representative of body parts and skin in general. That is, the present invention has therapeutic effects on various portions of the human body, such as legs, arms, the face, etc., and should not be limited in scope to any particular part of the body.

FIG. 2 provides a cross-sectional view of the skin 12, depicting the main regions of the skin 12, the epidermis 14, the derma 16, and the subcutaneous cellular level 18. The epidermis 14 is the outermost level of the skin 12. Generally, skin treatment products are designed to assist in forming a protective barrier on the epidermis 14 to retain water within the sublayers (i.e. the dermis 16 and the subcutaneous level 18) of the skin 12.

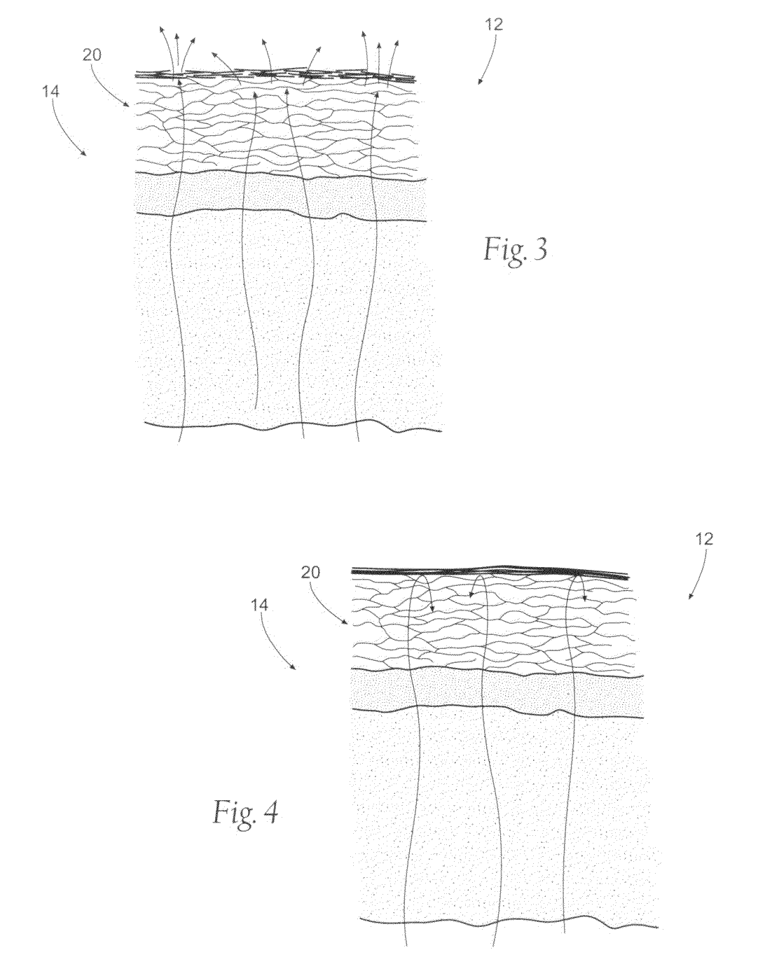

FIGS. 2, 3 and 4 provide cross-sectional views of the skin 12, focusing on the layers of the epidermis 14. The uppermost layer of the epidermis 14 is the stratum corneum 20, which contains the barrier function of the skin and keeps the skin hydrated. FIG. 3 shows a damaged stratum corneum 20, with water escaping through the skin, thereby hindering the natural process and ability of the skin 12 to stay properly hydrated. FIG. 4 shows a healthy stratum corneum 20, with water being retained within the skin 12, thereby keeping the skin 12 hydrated. This water flux from deeper skin layers, i.e. the derma 16 and the subcutaneous level 18, through the stratum corneum 20 (the uppermost layer of the epidermis 14) to the outside is called transepidermal water loss (TEWL). Measuring TEWL is one of the most important biophysical parameters for evaluating the efficiency of the human skin water barrier. Emollients and creams are used form an occlusive layer on the skin surface, reducing transepidermal water loss (TEWL), thus providing a temporary restoration of barrier function in skin.

In addition to physical or chemical measures for protection against UV-light, supplementation with antioxidants for preventing premature skin ageing can be used. These can be supplied to the skin via a diet rich in fruit or vegetables, or through additional oral or topical administration. Example 2 evaluated the antioxidant status of sodium PAGA using a commercially available kit from Randox™.

### Example 1

The present invention provides sodium PAGA compounds and compositions to reduce TEWL from the skin 12. As shown in FIG. 6, a portable device 22, known as the VapoMeter, was utilized to measure TEWL. The VapoMeter 22 has a closed chamber with a humidity sensor in which the water vapor pressure gradient is measured in g/h m² according to Fick's law. The VapoMeter is produced by Delfin Technologies Ltd. (Kuopio, Finland), and is a portable and battery-operated device containing a Honeywell humidity sensor in a closed chamber. The closed chamber conditions are created upon skin contact with a surface area of 1 cm diameter. Measuring time is between 8 and 10 s.

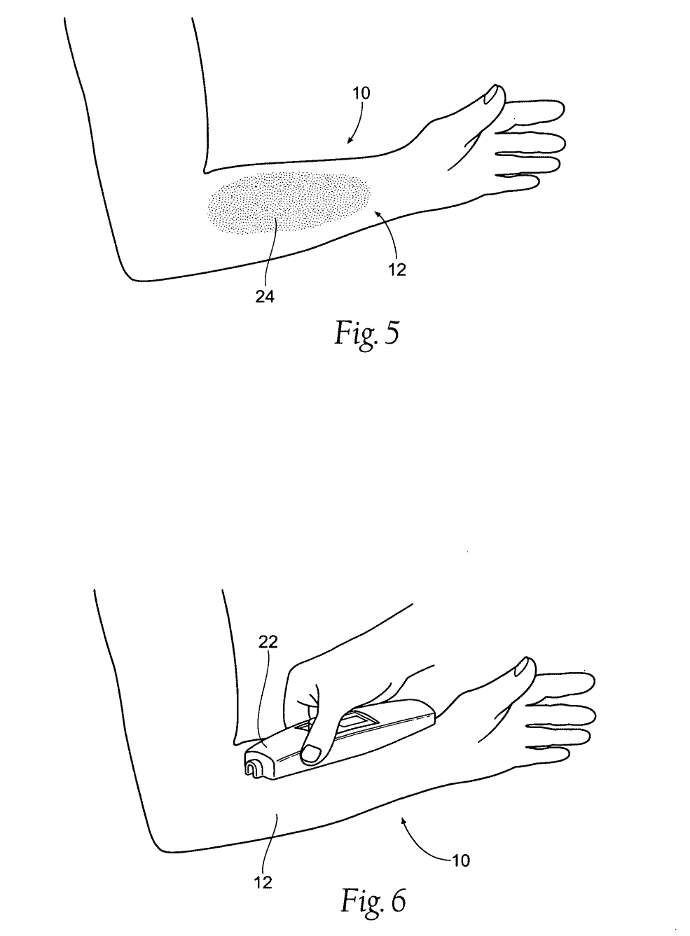

As depicted in FIG. 5, the skin 12 of the arm 10 is subjected to a topical treatment 24, containing a form of PAGA, referred to as microdispersed oxidized cellulose (M-DOC). Several people were tested. Volunteers were healthy males (6) and females (4) in the age range of 23-35 years old and without any skin disease. Subjects were instructed not to use creams, lotions or other personal skin care products before experiment and also not to expose their forearms to direct sun and to keep the test areas dry during the six hour testing period. The inner sides of the volar forearms were selected as measuring zones. This skin area is relatively hairless, preventing hairs or particles from touching the sensors. During the experimental study, room conditions were 24.0° C.±0.8% for temperature and 49.3±8.3% for relative humidity (RH).

On one of the four test zones (2 cm×2 cm) on the left or right forearm—randomized among the volunteers—the following measurements were carried out:

(a) Baseline (dry control) TEWL values by VapoMeter (expressed in g/h m²).

(b) Effect of a single topical application of sodium PAGA (1% w/v m·doc in dH₂O).

(c) Effect of a single topical application of sodium PAGA (2% w/v m·doc in dH₂O).

(d) Effect of a single topical application of sodium PAGA (5% w/v m·doc in dH₂O).

For (b), (c) and (d) the m·doc solution (100 μl) was pipetted onto test area, rubbed in gently, followed by a 30 minute rest period before first VapoMeter reading. VapoMeter measurements were taken at t=30, 60, 120, 180, 240, 300 and 360 minutes.

Results

The results are listed below in Table 1.

TABLE 1
Mean TEWL values (n = 10) ± SEM of forearm
after a single application of 2% and 5% sodium
PAGA in deionized water compare against a dry
control application (i.e. no application of sodium
PAGA) over 360 minutes

| | | 2% sodium PAGA | 5% sodium PAGA |
|---|---|---|---|
| | Control | solution in dH₂0 | solution in dH₂0 |
| | (g/h m²) ± | (g/h m²) ± | (g/h m²) ± |
| Time (min) | SEM | SEM | SEM |
| 30 | 10.0 ± 1.1 | 7.8 ± 0.8 | 7.5 ± 0.9 |
| 60 | 11.8 ± 1.3 | 9.6 ± 1.3 | 7.7 ± 1.0* |
| | | | (p < 0.05) |
| 180 | 11.7 ± 1.8 | 8.3 ± 0.8 | 8.3 ± 0.9 |
| 240 | 12.2 ± 1.6 | 9.3 ± 0.6 | 8.3 ± 0.8 |
| 300 | 12.2 ± 1.8 | 9.6 ± 0.7 | 9.7 ± 1.1 |
| 360 | 11.0 ± 1.3 | 10.0 ± 0.7 | 8.3 ± 0.5 |

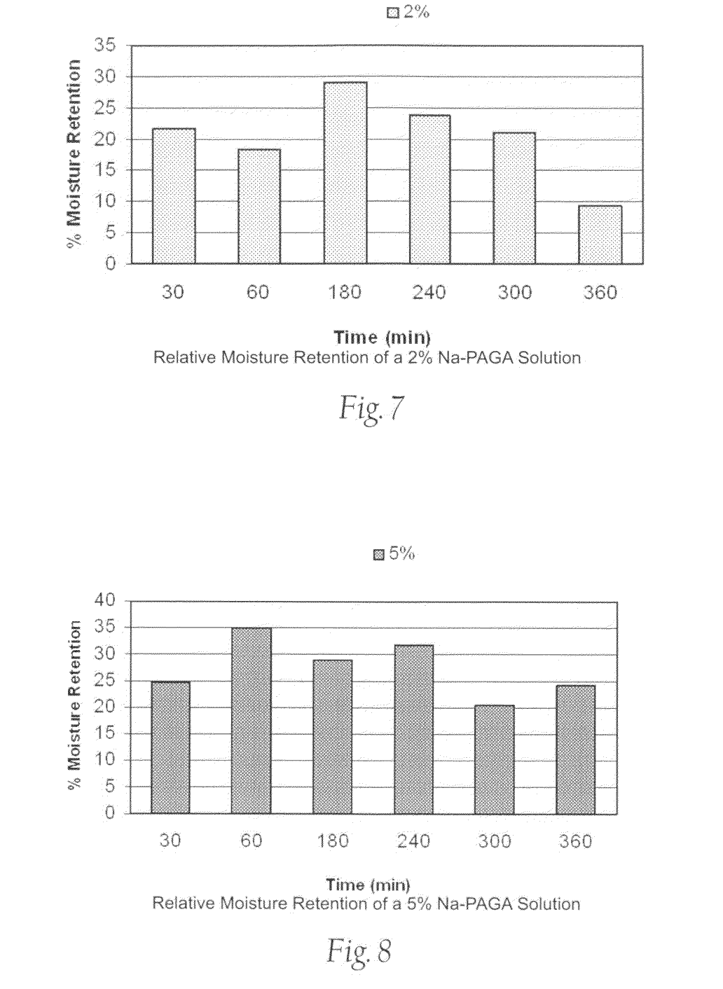

As shown in Table 1, the sodium PAGA solutions had favorable results compared to the control application. That is, there was less water loss through the epidermis at each time interval for both the 2% sodium PAGA solution and the 5% sodium PAGA solution when compared to the control application. This is also shown graphically in FIGS. 7 and 8, wherein the values of both the 2% solution (FIG. 7) and the 5% solution (FIG. 8) are represented as percentages of retention compared to the control application, which was taken at 0%. That is, the values listed in FIGS. 7 and 8 are the percentages of water retention for each of the samples above that of the control application. For example, after 60 minutes the 5% solution (FIG. 8) demonstrated 35% more water retention than that of the control application.

### Example 2

The Randox total antioxidant status assay is based on the reaction between metmyoglobin and hydrogen peroxide, which generates a free radical. This has a relatively stable blue-green color, which is measured at 600 nm. Antioxidants in the added sample cause suppression of this color production to a degree which is proportional to their concentration. Assay was carried out as per modified manual at 37° C. in a 96 well plate reader. Briefly equal volumes of sample (dissolved in deionized water) and chromogen are mixed well, brought to 37° C. and initial absorbance values were read (A₁). Following this the substrate was added and the reaction mixture was incubated for exactly 3 minutes before the second set of absorbance values were read (A₂).

Results

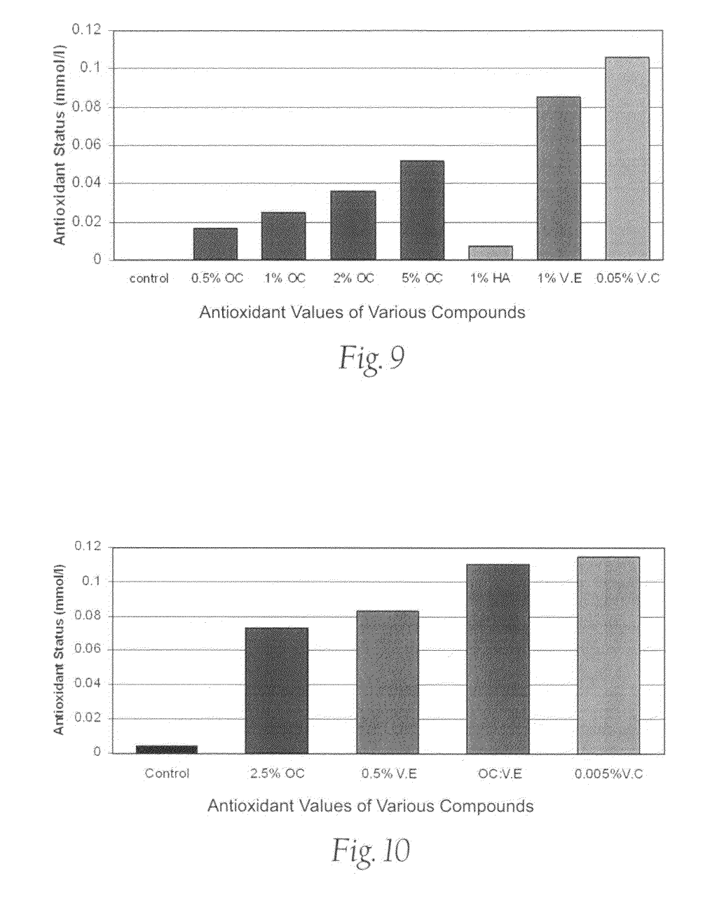

FIG. 9 shows the antioxidant status of various polyanhydroglucuronic acid, (PAGA) oxidized cellulose salt (sodium PAGA compounds) of the present invention, 0.5% OC, 1.0% OC, 2.0% OC, and 5.0% OC, compared to other known antioxidants currently used, such as hyaluronic acid (1% HA), vitamin E (1% V.E.), and vitamin C (0.05% V.C.), as well as being compared to a control (i.e. non-treated skin). The given concentrations are concentrations as dissolved in deionized water. As demonstrated in FIG. 9, the antioxidant capability of the sodium PAGA compounds has a correlation to the percentage of the sodium PAGA material within the compound.

FIG. 10 further demonstrates antioxidant status of the sodium PAGA compounds of the present invention (2.5% OC) compared to Vitamin E (V.E.) and Vitamin C (V.C.) compounds, as well as a control application. The given concentrations are concentrations as dissolved in deionized water. Also, FIG. 10 shows a compound comprising both vitamin E and sodium PAGA compound (O.C.:V.E.), which shows effective antioxidant status for the combination compound.

As demonstrated above, the oxidized cellulose compounds of the present invention have applicability in treating the skin with respect to hydrating the skin and delivering antioxidant protection to the skin. Section III below will further discuss the therapeutic qualities of the oxidized cellulose compounds of the present invention, discussing the effects of the compounds with respect to collagen expression.

III. Collagen Expression of the Skin

As discussed previously, wrinkles on the skin are caused by a diminished amount of collagen in the skin. The present invention contemplates oxidized cellulose compounds that can assist in collagen renewal in the skin. The following example discusses such collagen expression.

### Example 3

### Culture of HS-68 Cells

HS-68 cells were maintained in D-MEM medium supplemented with 10% (v/v) foetal calf serum (FCS), penicillin streptomycin (100 μg/ml) and incubated at 37° C., 5% CO₂ and 95% air in a Form a Scientific incubator. Cells were cultured in 50 ml of tissue culture medium in 175 cm² sterile vented flasks. Cells were gently washed with 10 ml of sterile phosphate buffered saline solution (PBS) prior to detachment from the culture flasks by incubation with 0.025% (w/v) trypsin/EDTA solution. HS-68 cells were then resuspended in culture medium at the desired cell number in culture media.

The HS-68 cells were plated in 96 well plates at concentrations ranging from 0.3×10⁴ cells per well to 2.2×10⁴ cells per well (depending on the length of the experimental incubation) and allowed to adhere overnight. Cells were then incubated with D-MEM medium supplemented with various PAGA derivative treatments and FCS concentration was reduced from 10% to 5% (v/v). HS-68 cells were incubated for 4 to 72 hours. Following the incubation period media was collected and stored at −20° C. until required for testing. Collagen and elastin assays were performed on the medians described below. Total cellular protein from HS-68 cells was determined described below.

Collagen secretion from HS-68 cells was determined using a Sircol™ soluble collagen assay kit purchased from Biocolor Ltd., (N. Irl.) following the methods described in the manual. Briefly, 100 μl test samples underwent a salt precipitating step to precipitate the collagen out of solution. The resulting solutions were then mixed with 1 ml of a Sircol Dye reagent for 30 minutes. After centrifuging and re-solubization of the pellet in 1 ml Alkali reagent, the absorbance was read at 540 nm in a platereader.

Mode of Action of the Sircol Dye Reagent with Soluble Collagens

The Sircol dye reagent contains Sirius Red. Sirius Red is an anionic dye with sulphonic acid side chain groups. These groups react with the side chain groups of the basic amino acids present in collagen. During the assay, the elongated dye molecules become aligned parallel to the long, rigid structure of intact triple helix collagens. This allows for the binding of the dye to collagen.

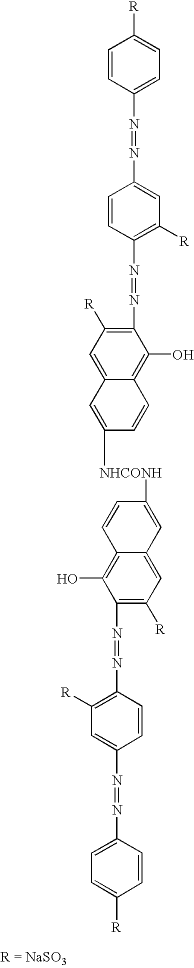

The Molecular Structure of the Sircol Dye

Collagen and elastin secretion were then normalized to the amount of protein in a well of a 96 well plate. After media was removed from wells, HS-68 cells were lysed with RIPA lysate buffer supplemented with EDTA and protease inhibitor. Samples were subjected to total protein assay with a bicinchoninic protein assay kit (BCA; Pierce, Rockford, Ill.). The purple-colored reaction product of this assay is formed by the chelation of two molecules of BCA with one cuprous ion. This water-soluble complex exhibits a strong absorbance at 562 nm.

Results

Modulation of Collagen Secretion from Fibroblast Cells by Na PAGA

Results from cellular viability studies showed that Na PAGA at 0.1% had the least detrimental effect on fibroblast viability. The effect of 0.1% Na PAGA on collagen secretion by fibroblasts was examined after 4, 8, 12, 24, 48, 72 hour incubations. After a 4 hour incubation 0.1% Na PAGA promoted collagen secretion from fibroblasts by 408% (p<0.005). There was also a statistically significant promotion in collagen secretion after 12 hours (168%; p<0.01) and 24 hours (216%; p<0.05).

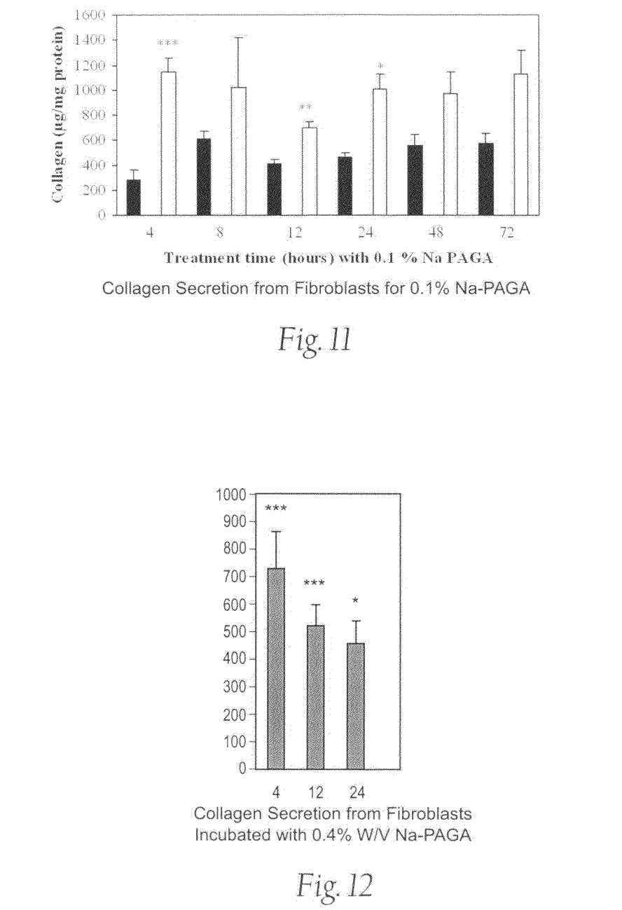

The results are shown graphically in FIG. 11, showing collagen secretion from fibroblasts incubated with 0.1% w/v Na PAGA for 4 to 72 hours. The results are compared to control applications. Control experiments were run in parallel; fibroblasts were incubated with DMEM supplemented with 5% FCS only. Control groups are represented as black bars and treatment groups as white bars on the graph. Data are expressed as μg/mg protein and are mean±SEM of duplicate values from three independent experiments. The values for *p<0.05, **p<0.01 and ***p<0.005 were compared with the control group. As shown, the compounds of the present invention showed improved collagen levels at each time interval compared to the control groups.

### Example 4

### Comparison of Collagen Secretion from Fibroblast Cell by Na PAGA and Other Industry Standards

The effect of Na PAGA on collagen secretion was measured and compared to the effect of known skin care products on collagen secretion using the Sircol™ soluble collagen assay kit. There was a statistically significant promotion in collagen secretion after 4, 12 and 24 hours with 0.4% Na PAGA, however there were no such significant increases with any other of the samples tested.

As shown in FIG. 12, collagen secretion from fibroblasts incubated with 0.4% w/v Na-PAGA for 4, 12 and 24 hours was tested and recorded. Control experiments were run in parallel; fibroblasts were incubated with DMEM supplemented with 5% FCS only. Data are expressed as percent of the control (the control bar is not show, but valued at 100%) and are mean±SEM of duplicate values from three independent experiments. *p<0.05 and ***p<0.005 were compared with the control group. At each of the time intervals, the Na-PAGA compounds of the present invention showed significant results compared to the control experiments.

IV. Delivery Methods

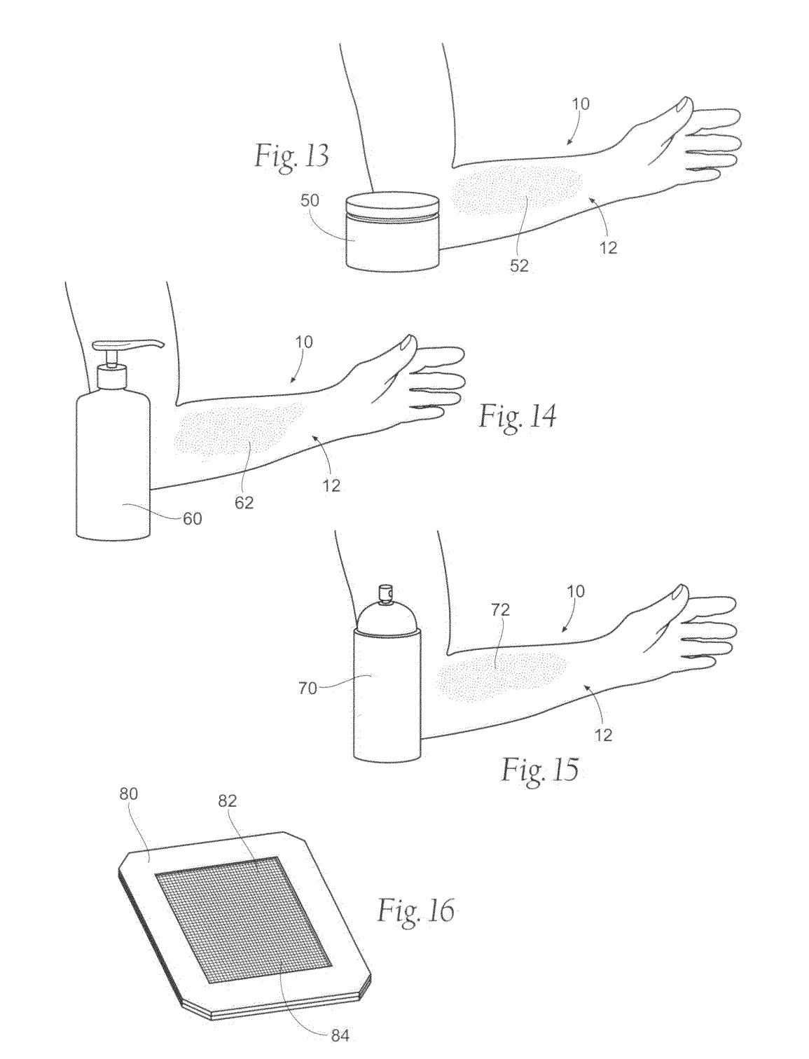

As previously discussed, the oxidized cellulose (OC) material of the present invention can be incorporated into various products and solutions to deliver the OC material. FIGS. 13-16 provide examples of various delivery methods for the OC material.

FIG. 13 depicts a container 50 containing a cream 52. The cream 52 contains the present OC material, preferably the discussed sodium PAGA material. The cream 52 can be applied to the skin 12 of the arm 10 to form a protective barrier. Likewise, FIG. 14 depicts a pump container 60 containing a lotion 62, that also can be applied to the skin 12 of the arm to form a protective barrier. FIG. 15 provides an aerosol can 70 containing a spray 72 that contains the material of the present invention.

FIG. 16 depicts an alternate used of the OC material of the present invention being incorporated into nanostructured devices. Nanotechnology and nanomaterials are increasing in use, such as the use of thin film technology for coatings in mass spectrometry technology. As an example, FIG. 16 represents a plate 80 used for spectrometry purposes. The plate 80 supports a substrate 82, with the OC materials comprising a thin film layer 84 covering the substrate 82. The film layer 84 can enhance the properties of the mass spectrometry process, by providing enhanced water resistance characteristics of the process, or possibly providing improved mechanical and optical properties for the process.

FIGS. 13-16 demonstrate potential delivery systems and uses for the present invention. However, other delivery systems can include oils, gels, nanofibers, non woven textiles, flat form, balms and other mediums. It is understood that the present invention should not be limited to any specific embodiment, but can be incorporated into numerous uses and systems.

V. Production of Oxidized Cellulose Compounds

The term “suitable” polysaccharide refers throughout to a polysaccharide that due to its chemical nature can serve as starting raw material for preparing PAGA.

The following section discusses the process for preparing the oxidized cellulose (OC) product, for which its potential therapeutic qualities have been demonstrated above. The process has significant advantages over other known processes, in particular over the process described in WO98/33822, which is also referred to as an oxidative hydrolysis process.

The method of the invention involves the transformation of oxidized cellulose (OC) to their salts from an excess of water soluble hydroxide, salt or organic base such as for example NaOH, KOH, LiOH, NH₄OH, Na₂CO₃, and R—NH₂. Oxidized cellulose in hydroxide (salt, organic base) solution is fully dissolved and a corresponding salt of OC is formed. In the case of double or mixed salts ion-exchange is performed using inorganic and/or organic water soluble salts or bases (such as chloride, nitrate, carbonate, sulphate etc.) with a different type of cation. Part of the original ions is thereby changed to an OC salt of the new cation. The ion exchange is fully controlled by the amount of inorganic and/or organic salt used. Oxidation, precipitation, washing, dehydration in water miscible or partially miscible organic solvents when necessary, and drying are subsequently performed.

The invention involves a more homogenous reaction system in contrast to the fully heterogeneous system employed in other oxidative hydrolysis systems. All the steps involved can be carried out at temperatures between −10 and 50° C., preferably at 25° C.

In addition, an admixture of an organic solvent (such as EtOH, isopropanol or other water miscible alcohol or organic solvents) is added to the aqueous system before or during hydrolysis, thereby shifting the range of applicable reaction temperatures down to below 50° C. and decreasing the heat capacity of the reaction system to achieve energy savings.

By way of an example, the reaction scheme for preparation of for example a sodium/calcium salt is outlined as follows:
1. Hydrolysis (sodium salt of OC is formed)

NaOH+R^{oc}—COOH→(R^{oc}—CO(X)Na⁺+H₂O;
2. Ion exchange (mixture of both salts is formed=MDOC Ca/Na)

NaOH+CaCl₂→2NaCl+Ca(OH)₂

2(R^{OC}—COO)Na⁺+Ca(OH)₂→(R^{OC}—COO)₂Ca²⁺+NaOH;
3. Oxidation (oxidation of residual terminal CH2OH and/or carbonyl groups, which are present in input raw material)

R⁰⁰CH₂OH+2H₂O₂→R^{OC}—COOH+3H₂O

R^{OC}—CH═O+H₂O₂->R^{OC}—COOH+H₂O; and
4. Adjustment of pH (removal of excess of alkaline entities)

NaOH/Ca(OH)₂+3HCl→NaCl+CaCl₂+3H₂O.

Oxidised cellulose is a copolymer of:

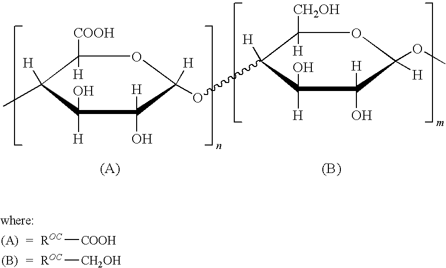

The invention results in high yields, uniform color of the final product and negligible inorganic carbonates content, as there is no source of carbonates in the reaction process. Unreacted inorganic carbonates are undesirable side products which are formed using other known methods. Moreover the risk of the presence of raw cellulose and/or unreacted oxidized cellulose fibers, which may have adverse effects for some applications of the product, in particular for internal applications, is decreased because the mixture can be filtered after the neutralization due to its being homogenous.

The process provides a number of advantages over other known processes for preparing oxidized cellulose including shorter production times (less than 7 hours) and lower production costs resulting in greater energy savings. In addition the content of cations in the final product is better controlled using appropriate weighing of the salts used. The method also results in a lighter color of the powder being formed thus enhancing the appearance of final products and in a narrower particle size distribution. The latter is especially important for aerosol packaging of the product.

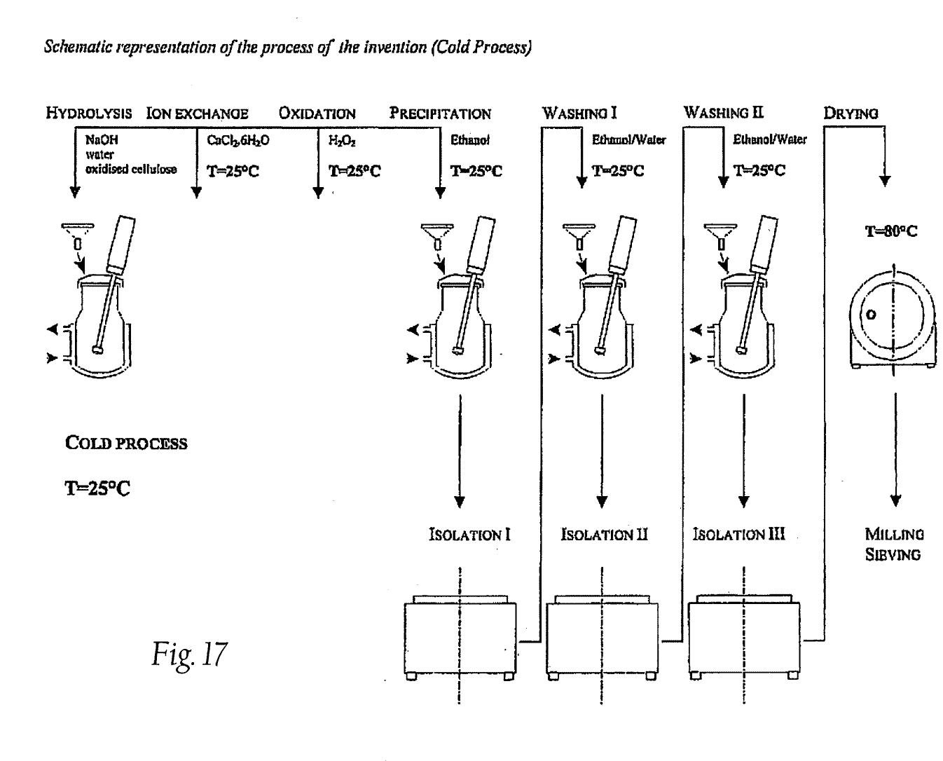

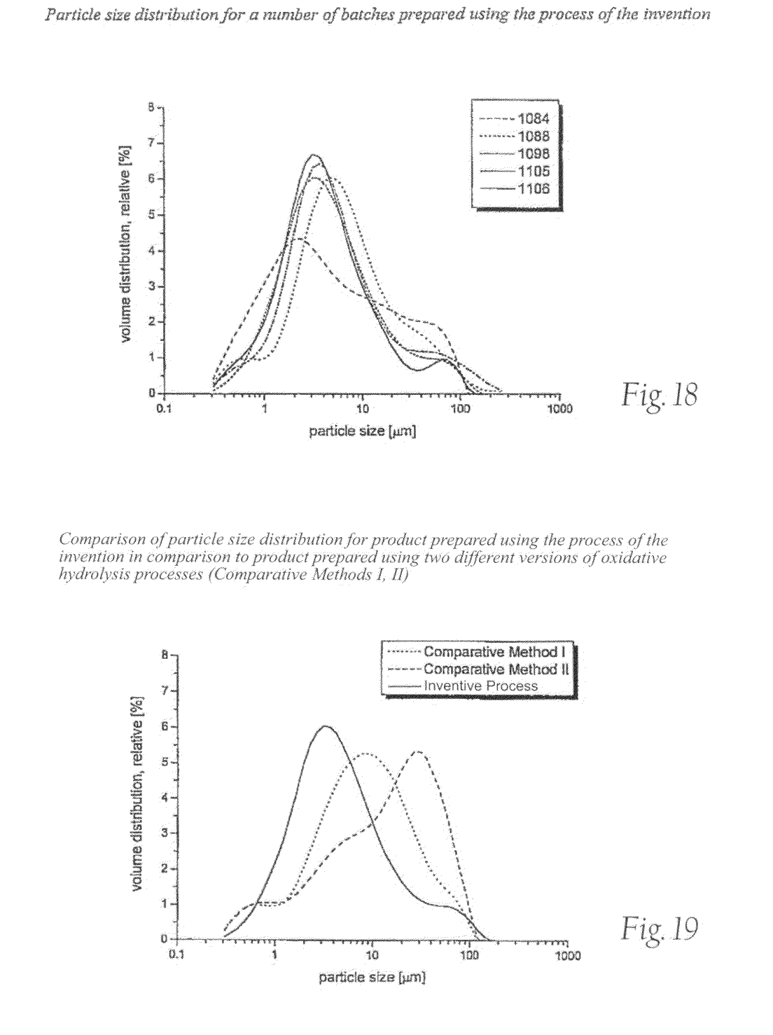

FIG. 17 provides a schematic outline of the process invention. Hydrolysis takes place at temperatures between −10 to +50° C., preferably at 25° C. Ion exchange and oxidation take place at the same temperature. The pH of the reaction mixture is adjusted to a value between 2.0 to 9.5 using mineral or organic acid depending on the pH required for the final product. After adjusting the pH, the reaction mixture is precipitated by a water-miscible organic solvent and the isolated filter cake is treated by repeated washing as required in suitable water/organic solvent mixtures for removal of residual inorganic salts. The solvent mixtures may involve EtOH/water, concentrated alcohol such as ethanol, isopropanol, or ether such as methylal, dimethylether, or other, similar compounds, depending on the required particle size for the final product. Finally the isolated wet product is dried at 25 to 100° C. depending on dryer used or milled and/or sieved if required or used directly from the above mixtures.

As a result, fibers of oxidized cellulose are transformed into microdispersed oxidized cellulose (such as a sodium-calcium salt of OC). This microdispersed form having haemostatic, antibacterial and/or other useful properties may be used in further processing (such as with spray or plaster, etc., manufacture).

The OC prepared by the method of the invention may be used in pharmaceutical or cosmetic compositions as described above and also in WO98/33822 the entire contents of which are herein incorporated.

The invention will be more fully understood from the following examples and description.

### EXAMPLES

### Example 5

In this example, cotton linters containing 99.1% w/w of α-cellulose and oxidized in 60% w/w nitric acid with an admixture of 3.8% nitrous acid were used in preparing a sodium salt of microdispersed polyanhydroglucuronic acid. The process was carried out at a temperature of 30° C. max, similarly to the process described in GBP 709684, which is incorporated by reference. The composition of the raw material is shown in Table 2.

TABLE 2
Composition of Raw Material in Example 5

| | |
|---|---|
| carboxyl groups | 16.3% w/w |
| carbonyl groups | 3.1% w/w |
| bound nitrogen | 0.5% w/w |

7 liters of water and 280 g of sodium hydroxide were placed into a 20 L jacketed glass-vessel and was stirred with an Ultra-Turrax stirrer T65 (IKA Werke CmbH & Co. KG, Germany). After dissolving of the sodium hydroxide, the above defined oxidized cotton linters (Table 2), which also contained about 10% of volatile matter (approximately 1.5 kg) were added to the vessel while constantly stirring for at least 30 minutes. The temperature was maintained at 35° C. max. Hydrogen peroxide (250 g) was then added, and the mixture was continuously stirred for 60 minutes, maintaining a constant temperature.

Next, the oxidation pH was adjusted to between 5 and 5.5 by adding concentrated HCl to the mixture. Then 10 L of 93% ethanol were added stepwise during about 10 minutes and the resulting colloid dispersion solution was then filtered. The obtained filter cake was dispersed into a 55% water-ethanol mixture and another filtration of the residue was redispersed into 5 L of pure ethanol and allowed to stand for at least 2 hours. The above steps are analogous to the process discussed in application WO 2007/026341, which is incorporated herein by reference, Isolated filter cake was again redispersed into 5 L isopropanol, and the mixture was allowed to stand for at least 10 hours. The final prepared suspension was filtered and dried in a vacuum try drier at the temperature of 70° C. and milled.

An analysis of the product obtained yielded the following results, shown in Table 3:

TABLE 3
Results of Product Produced According to Example 5

| | |
|---|---|
| loss on drying | 5.6% w/w |
| carboxyl group content | 17.5% w/w |
| bound nitrogen | less than 0.5% w/w |
| sodium content | 7.8% w/w |
| specific surface area | 87 m² · g⁻¹ |
| bulk volume | 8 ml/g |

The PAGA sodium salt, prepared according to Example 5, can be used directly as a hydrating agent in creams, lotions, balsams or ointments, as discussed above.

### Example 6

### MDOC

The process described below is used to form another oxidized cellulose product, a microdispersed oxidized cellulose product, referred to as MDOC or M-DOC. 600 L of demineralized water was added to a reactor. 28 kg of sodium hydroxide was stirred into the reactor and allowed to cool down to form a solution at 20-25° C., while stirring. While stirring the calcium chloride solution, 140 kg of raw oxidized cellulose (OC) (Example 5) was added progressively to the solution while stirring constantly, with the temperature being held between 20 and 25° C. The raw OC material was dried out at 80° C. for about 3 hours prior to addition to the reactor. The suspension in the reactor discolors during the addition of the OC, resulting in a yellow hue. After the final addition of the OC, stir for at least 30 minutes more to thoroughly mix the OC into the solution. The final reaction mixture will then be.

A solution of Calcium Chloride hexahydrate was prepared in a separate polyethylene container by combining 80 L of demineralized water with 54 kg of Calcium Chloride, hexahydrate. For approximately 40 minutes, add progressively the entire Calcium Chloride solution using a graduated vessel, while stirring. Once added, stir the reaction mixture for another 45 min at a temperature between 20 and 25° C.

Hydrogen peroxide H₂O₂ (35 L) will slowly be added using a graduated vessel to the reaction mixture for approximately 30 min. The temperature of the reaction mixture should not exceed 25° C. Stir the suspension for a further 30 min at a temperature between 20 and 25° C. The suspension discolors to a snowy white hue.

The pH of the reaction mixture will be adjusted by slowly adding hydrochloric acid (HCl) until a pH between 4.0 and 4.8 is attained. Stir the mixture for 15 minutes after each HCl addition.

1000 L of Ethanol is added to the reactor using a graduated vessel, while continuously stirring. After the ethanol is added to the reactor, stir the formed suspension for another 15 minutes at a constant temperature between 20 and 25° C. Isolate the raw product by centrifuging the parent liquor off. Disperse the isolated filter cake using a Turrax stirrer in 400 L of water.

Gradually supply 650 L of ethanol to the reactor using a graduated vessel, while continuously stirring for 30 minutes with the Turrax stirrer. Stir the formed suspension for another 30 min at a constant temperature between 20 and 25° C. The raw product will then be isolated by centrifuging the parent liquor off. Disperse the isolated filter cake using the Turrax stirrer and adding 800 L of Ethanol and stir the suspension for at least 120 minutes. Isolate the raw product by centrifuging the parent liquor off. Disperse the isolated filter cake using the Turrax stirrer in 800 L of isopropyl alcohol. Isolate the raw product by centrifuging the parent liquor off and dry the filter cake in a tray drier at a temperature of 80° C.

After 15 hours drying submit a sample for quality control to check the content of the dry matter. The drying is finished when the content of dry matter exceeds 93%. Mill the dried powder using an Alpine mill.

An analysis of the product obtained yielded the following results, shown in Table 4:

TABLE 4
Results of Product Produced According to Example 6

| | |
|---|---|
| loss on drying | 7.6% w/w |
| carboxyl group content | 20.1% w/w |
| bound nitrogen | 0.5% w/w |
| sodium content | 2.3% w/w |
| calcium content | 6.1% w/w |
| pH of 1% water solution | 5.67 |
| bulk volume | 10.1 ml/g |

The prepared PAGA calcium sodium salt (MDOC) can be used directly as a haemostatic agent in various products, as discussed above. The size of the particles produced according to Example 6 generally had a diameter less than 50 μm. Generally speaking, the particle size of the compounds formulated according to the present invention can be modified. Depending on the water content remaining in the final filter cake, the particle size will be larger (more water retained in the particles) or smaller (less water retained in the particles. Both larger and smaller particles have applicability, whereas larger, granular particles may be incorporated into wound dressings, while smaller, fine particles can be more easily incorporated into such products as sprays and ointments.

### Example 7

### PAGA Na/Zn

Another form of PAGA is contemplated, containing a sodium/zinc mixture. The Paga Na/Zn mixture is discussed below.

550 ml of demineralised water was supplied to a reactor of nominal volume, i.e. a 2 L reactor. 18 g of sodium hydroxide (NaOH) was added to the water and the formed solution was cooled down to 20-25° C. while stirring. 100 g of oxidised cellulose (OC) (Example 5), previously dried at 90° C. for at least 5 hours (carboxyl content 18.2% w/w, produced according Example 5), was progressively added to the reactor, stirring constantly. During the addition, the temperature of reaction mixture was held between 25 and 30° C. The reaction mixture was stirred for at least 30 min after of the final addition of oxidised cellulose.

A water solution of 33.1 g of zinc chloride in 100 ml of demineralised water was gradually added to reaction mixture. Reaction mixture was then stirred for at least 80 min, with the temperature maintained between 25 and 30° C. Then 30 g of hydrogen peroxide was slowly and progressively added, and reaction mixture was stirred for another 30 min at temperature of 20-25° C.

The oxidation pH of reaction mixture was adjusted by adding Hydrochloric Acid (HCl) until a pH between 5.0 and 6.0 is attained. The mixture was stirred for min 15 minutes after each hydrochloric acid addition.

The reaction mixture was then precipitated by the addition of 1100 ml of ethanol. The formed suspension was further stirred for 10 minutes at a constant temperature between 20 and 25° C. The wet product was isolated by filtering the parent liquor off. The isolated filter cake was then dispersed in a mixture of 500 ml of distilled water and 800 ml of ethanol. Isolated wet product was again dispersed in 75% of water/ethanol mixture. The isolated raw product was dried in a try drier at the temperature of 70° C. for approximately 10 hours. The resulting dried product was milled using a standard kitchen mixer and sieved using a system consisting of two sieves (90 and 400 μm). The yield for the resulting product, polyanhydroglucuronic acid sodium/zinc salt (PAGA Na/Zn), is shown below in Table 5.

TABLE 5
Results of PAGA Na/Zn Mixture Obtained According to
Example 7

| | |
|---|---|
| loss on drying | 4.0% w/w |
| carboxyl group content | 17.8% w/w |
| bound nitrogen | less than 0.05% w/w |
| sodium content | 2.6% w/w |
| zinc content | 8.1% w/w |
| particle size | between 90 and 400 μm |

The resultant PAGA Na/Zn composition was tested against various microorganisms to determine the potential antimicrobial efficacy of the composition. The results are shown below in Table 6.

TABLE 6
Results of 24 hours tests (USP) - antimicrobial
efficacy:

| | Number of colonies in 1 ml | | |
|---|---|---|---|
| | of sample | | |
| | time | | |
| Microorganism | 0 | 6 hour | 24 hour |
| Staphylococcus aureus CCM | 5.E+06 | 6.E+03 | <1000 |
| 4516 | | | |
| Pseudomonas aeruginosa | 2.E+06 | 3.E+03 | <1000 |
| CCM 1961 | | | |
| Candida albicans CCM 8215 | 5.E+06 | 4.E+05 | 8.E+03 |
| Aspergillus niger CCM 8222 | 3.E+06 | 2.E+05 | 1.E+04 |

The results in Table 6 show that the compound had an antimicrobial effect against all the tested micro-organisms, most notably the Staphylococcus aureus 4516 and Pseudomonas aeruginosa 1961 micro-organisms, decreasing the colony to less than 1000 for both micro-organisms after 24 hours.

### Example 8

### Cytotoxicity Test

The above compounds of Example 5 (PAGA Na), Example 6 (MDOC), and Example 7 (Paga Zn/Na) were subjected to cytotoxicity testing.

Plates with a flat bottom were used for tissue cultures, and 8×12 holes (TPP) were used for cultivation. The 3T3 cellular line was used as cellular substrate. The tests were carried out by the suspension of 105 cells in 1 ml of culture medium, with an inoculation of 0.1 ml of suspension (1×104 cells) to each hole. The cultures were kept at 37° C. and contained 7.5% CO₂ (incubator for tissue cultures Heraeus).

Pre-cultivation for the cellular culture mono-layer was pre-cultivated 24 hours prior to exposure to the tested material. The cultures were placed in quadruplets for sample materials in concentrations of 100, 250, 500, 1000, 2500, 10 000 g/ml of DMEM without serum and control. Because of the high cytotoxicity, lower concentrations of these materials were tested at samples VZ089 and VZ090 (the VZ 089 sample in concentrations of 10, 25, 50, 100, 250, 500 and 1000 g/ml, the VZ090 sample in concentrations of 1, 5, 10, 25, 50 and 100 g/ml). After 24 hours of pre-cultivation the culture medium was removed from cultures placed in the quadruplets and 0.2 ml of sample material dilution, respective of control samples (PK, K), was added.

Then 24-hour cultivation upon the temperature of 37° C. in the atmosphere with 7.5% CO2 followed. After the cultivation end the cultivation was determined quantitatively (fluorimetrically) on the basis of vital dye incorporation (neutral red). The level of cytotoxicity was determined according to fluometrical determination of quantitative cell dying. The fluometrical method of cytotoxicity determination is based on vital dye (neutral red) incorporation into live cells (neutral red uptake) and detection of fluorescence in the system of exciting (530 nm) and emission (590 nm) filter upon cold light transmission. The fluorometer Wellfluor of the company Denley, U.K. was used for detection of outcoming fluorescence and measurement of fluorescent units. The level of cytotoxicity is expressed in % of detected fluorescence in the culture with tested substance presence in comparison with a control culture without tested substance presence. (Ref.: Rat, P.: New microtitration fluorimetric technology—Applications to dermotoxicity. Nouv. Dermatol. 12:471, 1993). Results of cytotoxicity testing are listed below in Table 7.

TABLE 7
Cytotoxicity of
Various Oxidized Cellulose Compounds

| | non-toxic |
|---|---|
| Sample | concentration [μg/ml] |
| PAGA Na | 2500 |
| MDOC | 1000 |
| PAGA Na/Zn | 10 |

### Example 9

### MDOC H+

The following example demonstrates a variation of the MDOC compound demonstrated in Example 6, referred to as MDOC H+. 300 g of MDOC powder (Example 6) and 3 L of 93% ethanol were thoroughly mixed in a jacketed-glass reactor using a Turrax T50 stirrer ((IKA Werke CmbH & Co. KG, Germany). 180 g of 65% w/w nitric acid was added and the mixture was stirred at 1000 rpm (T50) for at least 120 minutes. Then centrifugation of reaction mixture was done and approximately 700 g of isolated filter cake was again thoroughly dispersed in a mixture of HNO₃ (65% min)/ethanol 170 g/2000 g for 30 minutes. After isolation, the obtained filter cake was three times washed in 1 L of pure ethanol and dried in a laboratory drier at 50° C. for 22 hours. The resulting product contained a high ratio of non-saturated COOH groups, as shown below in Table 8. The higher the carboxyl content, the higher the bioresorption. However, vary high carboxyl contents can limit the flexibility and usefulness of the compounds. The carboxyl content of compounds of the present invention is preferably up to about 25% w/w, with a preferable range being between about 16-220.

TABLE 8
Results of Products Produced According to Example 9

| | | |
|---|---|---|
| loss on drying | 10.2% | w/w |
| carboxyl group content | 18.2% | w/w |
| bound nitrogen | 0.3% | w/w |
| sodium content | 1.2% | w/w |
| calcium content | 0.5% | w/w |
| pH of 1% water solution | 2.44 | |

Mixtures of formed salt MDOC H⁺ with MDOC (ratio in w/w) were tested on antimicrobial efficacy (24 hours test USP27), with ratios of 1:1, 1:2, 1:3, 1;4, 1:5, and 1:10 of MDOC+:MDOC being tested. The results are represented, below, in Tables 9-14.

TABLE 9
Efficacy of Compounds Having a Ratio of MDOCH⁺/MDOC
(1/1) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | <1000 | <1000 |
| 4516 | | | |
| Pseudomonas aeruginosa | 5.E+06 | <1000 | <1000 |
| CCM 1961 | | | |
| Candida albicans CCM 8215 | 6.E+06 | 3.E+05 | 5.E+04 |
| Aspergillus niger CCM 8222 | 1.E+06 | 1.E+05 | 2.E+04 |

TABLE 10
Efficacy of Compounds Having a Ratio of MDOCH⁺/MDOC
(1/2) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | <1000 | <1000 |
| 4516 | | | |
| Pseudomonas aeruginosa | 4.E+06 | 2.E+03 | <1000 |
| CCM 1961 | | | |
| Candida albicans CCM 8215 | 5.E+06 | 3.E+05 | 4.E+04 |
| Aspergillus niger CCM 8222 | 2.E+06 | 1.E+05 | 1.E+04 |

TABLE 11
Efficacy of Compounds Having a Ratio of MDOCH⁺/MDOC
(1/3) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | <1000 | <1000 |
| 4516 | | | |
| Pseudomonas aeruginosa | 4.E+06 | 3.E+04 | 2.E+03 |
| CCM 1961 | | | |
| Candida albicans CCM 8215 | 5.E+06 | 4.E+05 | 7.E+04 |
| Aspergillus niger CCM 8222 | 2.E+06 | 7.E+05 | 3.E+05 |

TABLE 12
Efficacy of Compounds Having a Ratio of MDOCH⁺/MDOC
(1/4) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | <1000 | <1000 |
| 4516 | | | |
| Pseudomonas aeruginosa | 4.E+06 | 7.E+04 | 5.E+03 |
| CCM 1961 | | | |
| Candida albicans CCM 8215 | 5.E+06 | 6.E+05 | 2.E+05 |
| Aspergillus niger CCM 8222 | 2.E+06 | 8.E+05 | 5.E+05 |

TABLE 13
Efficacy of Compounds Having a Ratio of MDOCH⁺/MDOC
(1/1) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | <1000 | <1000 |
| 4516 | | | |
| Pseudomonas aeruginosa | 5.E+06 | 9.E+03 | 3.E+03 |
| CCM 1961 | | | |
| Candida albicans CCM 8215 | 6.E+06 | 4.E+05 | 1.E+05 |
| Aspergillus niger CCM 8222 | 1.E+06 | 5.E+05 | 4.E+05 |

TABLE 14
Efficacy of Compounds Having a Ratio of MDOCH⁺/MDOC
(1/10) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | 2.E+04 | <1000 |
| 4516 | | | |
| Pseudomonas aeruginosa | 5.E+06 | 7.E+04 | 5.E+03 |
| CCM 1961 | | | |
| Candida albicans CCM 8215 | 6.E+06 | 3.E+05 | 8.E+05 |
| Aspergillus niger CCM 8222 | 1.E+06 | 1.E+05 | 7.E+05 |

### Example 10

### PAGA Ag

The following example provides another oxidized cellulose (OC) material according to the present invention, that incorporates the silver into the OC material.

300 ml of demineralised water was supplied to a reactor of nominal volume, 1 L. 10 g of sodium hydroxide (NaOH) was added and the formed solution was cooled down to 20-25° C. while stirring. 50 g of oxidised cellulose, having a carboxyl content of 18.0% w/w, prepared according to Example 5, above, and previously dried at 90° C. for at least 5 hours was progressively added to the reactor, stirring constantly. The temperature of the reaction mixture was maintained between 25 and 35° C. The reaction mixture was stirred at least 30 min after the last addition of oxidised cellulose to the mixture.

20 g of hydrogen peroxide (H₂O₂) was slowly and progressively added to the reaction mixture, and the reaction mixture was stirred for another 30 min at temperature of 20-25° C. After oxidation, an aqueous silver nitrate (40.0 g silver nitrate in 100 ml of demineralised water) was gradually added to the reaction mixture, which was then stirred for at least 105 minutes; maintaining a constant temperature. Due to the low solubility of the formed silver salt, the wet product was easily isolated from the water by centrifugation. The isolated wet product was again washed in a mixture of 250 ml of water and 10 ml of hydrogen peroxide. Then the product was dehydrated in ethanol and isopropanol, similarly to the other PAGA salts describe in previous examples. The PAGA silver salt was dried at 50° C. The final product analysis is shown below, in Table 15.

TABLE 15
Product Analysis of PAGA-Ag Compound Produced
According to Example 10

| | | |
|---|---|---|
| loss on drying | 3.7% | w/w |
| carboxyl group content | 20.9% | w/w |
| bound nitrogen | 0.7% | w/w |
| sodium content | 0.2% | w/w |
| silver content | 29.1% | w/w |
| pH of 1% water solution | 4.77 | |

Mixtures of PAGA silver salts formed according to Example 10 were combined with MDOC, in various ratios (ratio in w/w), and were tested on antimicrobial efficacy (24 hours test USP27. The results are represented, below, in Tables 16-20.

TABLE 16
Efficacy of Compounds Having a Ratio of PAGA Ag
Salt/MDOC (1/1) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | <1000 | <1000 |
| 4516 | | | |
| Pseudomonas aeruginosa | 5.E+06 | <1000 | <1000 |
| CCM 1961 | | | |
| Candida albicans CCM 8215 | 6.E+06 | <1000 | <1000 |
| Aspergillus niger CCM 8222 | 1.E+06 | <1000 | <1000 |

TABLE 17
Efficacy of Compounds Having a Ratio of PAGA Ag
Salt/MDOC (1/5) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | <1000 | <1000 |
| 4516 | | | |
| Pseudomonas aeruginosa | 5.E+06 | <1000 | <1000 |
| CCM 1961 | | | |
| Candida albicans CCM 8215 | 6.E+06 | <1000 | <1000 |
| Aspergillus niger CCM 8222 | 1.E+06 | <1000 | <1000 |

TABLE 17
Efficacy of Compounds Having a Ratio of PAGA Ag
Salt/MDOC (1/10) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 3.E+06 | <1000 | <1000 |
| 4516 | | | |
| Pseudomonas aeruginosa | 5.E+06 | <1000 | <1000 |
| CCM 1961 | | | |
| Candida albicans CCM 8215 | 6.E+06 | <1000 | <1000 |
| Aspergillus niger CCM 8222 | 1.E+06 | 2.E+03 | <1000 |

TABLE 18
Efficacy of Compounds Having a Ratio of PAGA Ag
Salt/MDOC (1/20) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | <1000 | <1000 |
| 4516 | | | |
| Pseudomonas aeruginosa | 4.E+06 | <1000 | <1000 |
| CCM 1961 | | | |
| Candida albicans CCM 8215 | 5.E+06 | <1000 | <1000 |
| Aspergillus niger CCM 8222 | 2.E+06 | 1.E+03 | <1000 |

TABLE 19
Efficacy of Compounds Having a Ratio of PAGA Ag
Salt/MDOC (1/50) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | 3.E+04 | <1000 |
| 4516 | | | |
| Pseudomonas aeruginosa | 4.E+06 | 7.E+05 | 8.E+04 |
| CCM 1961 | | | |
| Candida albicans CCM 8215 | 5.E+06 | 9.E+04 | 2.E+03 |
| Aspergillus niger CCM 8222 | 2.E+06 | 6.E+05 | 4.E+04 |

TABLE 20
Efficacy of Compounds Having a Ratio of PAGA Ag
Salt/MDOC (1/100) on Various Microorganisms

| | No of colonies in 1 ml | | |
|---|---|---|---|
| | of sample | | |
| | time | | |
| Microorganism | 0 | 6 hour | 24 hour |
| Staphylococcus aureus CCM | 7.E+06 | 1.E+05 | 6.E+04 |
| 4516 | | | |
| Pseudomonas aeruginosa | 4.E+06 | 9.E+05 | 1.E+05 |
| CCM 1961 | | | |
| Candida albicans CCM 8215 | 5.E+06 | 1.E+05 | 4.E+03 |
| Aspergillus niger CCM 8222 | 2.E+06 | 7.E+05 | 5.E+04 |

### Example 11

### Soluble MDOC

50 g of MDOC (Example 6) was thoroughly dispersed in 500 ml of distilled water using a turrax T25 stirrer (IKA Werke CmbH & Co. KG, Germany). Insoluble portion was removed by centrifugation at 4000 rpm for 2×30 minutes. The obtained supernatant was reprecipitated by 1500 ml of ethanol. Isolated filter cake was dehydrated in 2 L of 2-propanol and dried at 50° C. and milled. The final product was analyzed and compared to the MDOC product produced according to Example 6. The results are shown below in Table 21.

TABLE 21
Analytical Comparison of an MDOC Compound (Example 6)
Compared to a Soluble MDOC Compound

| | | soluble |
|---|---|---|
| TESTED PARAMETER | MDOC | MDOC |
| pH of water | 5.34 | 5.76 |
| extract | | |
| Loss on drying | 2.4 | 7.35 |
| Nitrogen content | 0.1 | 0.2 |
| Carboxyl content | 21.0 | 23.1 |
| Sulphate ash | 29.2 | 34.0 |
| Calcium | 6.6 | 8.5 |
| Sodium | 2.2 | 2.1 |

Solubility can be changed and altered, depending on the particular use of the material. For example, a non-soluble MDOC material has very good haemostatic efficacy, while a soluble MDOC material is useful in situations and products where hydration is desirable. Solubility is affected by the type of cation that is used for the compound. For example, PAGA compounds combined with Na, K, or Li ions exhibit good solubility, PAGA compounds combined with Zn, Mg, Ca, or Co ions have partial solubility, while PAGA compounds combined with Cu and Ag ions have very limited solubility.

The length of the PAGA chain will affect the solubility, as well. Shorter length chains will provide higher levels of solubility, while longer chains will be less soluble. Similarly, the carboxyl content can affect the solubility of the compound with a higher carboxyl content being more soluble. Depending on the specific use of the oxidized cellulose, the compounds of the present invention can be adjusted and adapted accordingly.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

### REFERENCES

1. Burchard W. Ed., Polysaccharide, Eigenschaften and Nutzung, Springer
Verlag, Berlin 1985, p. 144 2. Kenyon et al., Ind. Eng. Chem., 4_i, No 1, 2-8 (1949)
3. Painter J. et al., Carbohydrate Research 140, 61 (1985);
4. Alhaique F., Chim. Oggi 11-15, 17 (1986)
5. Kaversneva E. P., Doklady AN SSSR (U.S.S.R.) 78 (3), 481 (1951);
6. Nevell T. P., J. Text. Ind. 42, 91 (1951); 7. Sihtola M. et al., J. Polym. Sci, Part C, (2), 289 (1963);
8. Pasteka M., Chemicke Zvesti (Slovakia) (20), 855 (1966)
9. Saito T., Isogai A., Biomacromolecules 5 (5), 1983 (2004);
10. Li et al., Oxidaton of primary alcohols to carboxylic acids with a TEMPO catalyst using NaClO₂ and NaClO

I/We claim:

1. A compound used for stimulating collagen expression in the skin, comprising:
a micronized oxidized cellulose salt compound having a uniform particle size between about 0.0001 mm to 0.050 mm.

2. The compound according to claim 1 wherein the micronized oxidized cellulose salt comprises a metal salt.

3. The compound according to claim 2 wherein the metal salt is preferably selected from but not limited to the group consisting of: sodium, calcium, zinc, copper, and silver salts.

4. The compound according to claim 1, wherein said micronized oxidized cellulose salt compound comprises up to approximately 5% w/v of the stimulating compound.

5. The compound according to claim 1, wherein said micronized oxidized cellulose salt compound comprises a polyanhydroglucuronic acid salt.

6. The compound according to claim 5 wherein said polyanhydroglucuronic acid salt comprises a metal salt.

7. The compound according to claim 6 wherein the metal in said polyanhydroglucuronic acid metal salt is selected from the group consisting of: sodium, calcium, zinc, copper, and silver salts, and combinations thereof.

8. The compound according to claim 7 wherein said polyanhydroglucuronic acid metal salt comprises polyanhydroglucuronic acid sodium salt.

9. The compound according to claim 7 wherein said polyanhydroglucuronic acid metal salt comprises polyanhydroglucuronic acid sodium/zinc salt.

10. A method of stimulating collagen expression in the skin comprising the steps of:
applying said compound of claim 1 to the skin.

11. The method of claim 10 wherein said compound comprises a gel.

12. The method of claim 10 wherein said compound comprises an aerosol spray.

13. An antioxidant comprising:
a micronized oxidized cellulose salt compound having a uniform particle size between about 0.0001 mm to 0.050 mm.

14. The compound according to claim 13 wherein the micronized oxidized cellulose salt comprises a metal salt.

15. The compound according to claim 14 wherein the metal salt is selected from the group consisting of: sodium, calcium, zinc, copper, and silver salts, and combinations thereof.

16. The compound according to claim 13, wherein said micronized oxidized cellulose salt compound comprises up to about 5% w/v of the stimulating compound.

17. The compound according to claim 13, wherein said micronized oxidized cellulose salt compound comprises a polyanhydroglucuronic acid salt.

18. The compound according to claim 17 wherein said polyanhydroglucuronic acid salt comprises a metal salt.

19. The compound according to claim 18 wherein the metal in said polyanhydroglucuronic acid metal salt is selected from the group consisting of: sodium, calcium, zinc, copper, and silver salts.

20. The compound according to claim 19 wherein said polyanhydroglucuronic acid metal salt comprises polyanhydroglucuronic acid sodium salt.

21. The compound according to claim 13 further comprising a vitamin E compound.

22. A compound for reducing transepidermal water loss comprising:
a micronized oxidized cellulose salt compound having a uniform particle size between about 0.0001 mm to 0.050 mm.

23. The compound according to claim 22 wherein the micronized oxidized cellulose salt comprises a metal salt.

24. The compound according to claim 23 wherein the metal salt is selected from the group consisting of: sodium, calcium, zinc, copper, and silver salts, and combinations thereof.

25. The compound according to claim 22, wherein said micronized oxidized cellulose salt compound comprises up to about 5% w/v of the stimulating compound.

26. The compound according to claim 22, wherein said micronized oxidized cellulose salt compound comprises a polyanhydroglucuronic acid salt.

27. The compound according to claim 26 wherein said polyanhydroglucuronic acid salt comprises a metal salt.

28. The compound according to claim 27 wherein the metal in said polyanhydroglucuronic acid metal salt is selected from the group consisting of: sodium, calcium, zinc, copper, and silver salts.

29. The compound according to claim 28 wherein said polyanhydroglucuronic acid metal salt comprises polyanhydroglucuronic acid sodium salt.

30. A method of reducing transepidermal water loss through the skin comprising the steps of:
applying said compound of claim 22 to the skin.

31. The method of claim 30 wherein said compound comprises a gel.
